(19) United States
(12) Patent Application Publication
(10) Pub. No.: US 2010/0121295 A1
(43) Pub. Date: May 13, 2010

(54) Products comprising polymeric webs with nanoparticles

(75) Inventors: Dimitris Ioannis Collias, Mason, OH (US); Norman Scott Broyles, Hamilton, OH (US); Terrill Alan Young, Cincinnati, OH (US); Susan L. Wilking, Hamilton, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company

(21) Appl. No.: 11/413,546

(22) Filed: Apr. 28, 2006

(51) Int. Cl. A61F 13/15; C08K 5/541

(52) U.S. Cl. 604/367; 524/262; 977/773

(57) ABSTRACT

A product comprises an expanded polymeric web includes of between about 0.1 and about 70 weight percent of a compound comprising nanoparticles. The expanded polymeric web includes between about 30 and about 99.9 weight percent of a generally melt processable polymer. The web also includes between about 0.0 and about 50 weight percent of a compatibilizer.

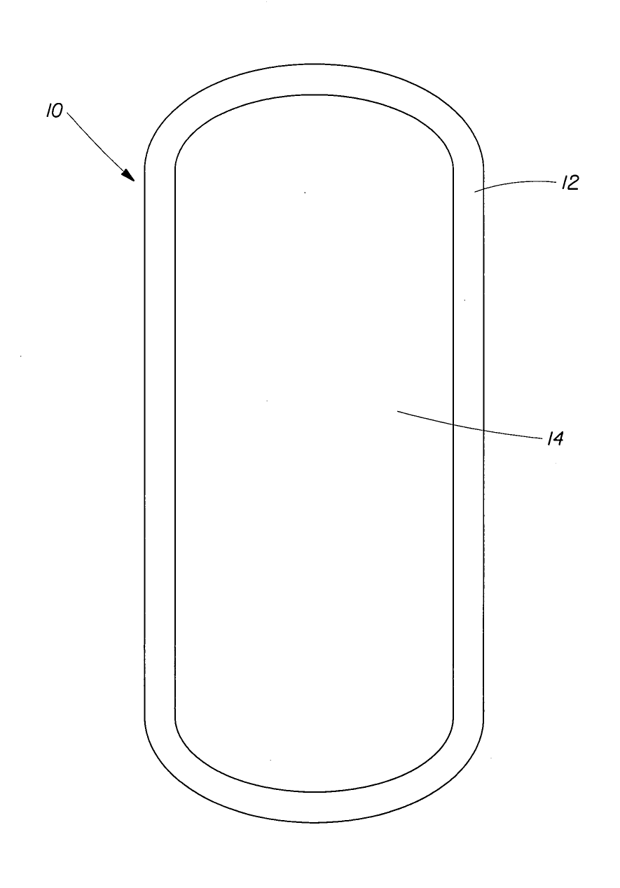

### FIELD OF THE INVENTION

The present invention relates to products comprising polymeric webs comprising nanoparticles. The invention relates particularly to products comprising expanded polymeric webs comprising nanoparticles.

### BACKGROUND OF THE INVENTION

Fillers (also called extenders) are used in the plastics industry (e.g. blow molded bottles, injection molded parts, blown or cast films, and fibers or non wovens) to “fill” the plastic parts. The purpose of the filler can be multifold. The filler can be used to replace plastic at lower cost thus improving the overall cost structure of the parts. The filler can also be used for performance related reasons such as stiffening, creating porosity, altering surface properties, etc. Typical examples of fillers are clays (natural and synthetic), calcium carbonate, talc, silicate, glass microspheres (solid or hollow), ceramic microspheres, glass fibers, carbon-based materials (platelets, irregular, and fibril), etc.

To achieve their function, fillers need to be dispersed homogeneously in the polymer matrix and have optimal adhesion with the polymer matrix. These properties of homogeneous dispersion and optimal adhesion are achieved with good dispersive and distributive mixing and surface modification of the filler particles, such as coating of the surface of calcium carbonate fillers with stearic acid. Also, the surface modification alters the surface energy of some of the fillers, thus allowing optimal mixing with the polymer matrix. The typical size of the individual filler particles is on the order of μm or tens of gm, which results in <1 m²/g specific surface area available for interaction with the polymer matrix. This small specific surface area may explain the limited benefits typically seen with fillers.

Using a filler material having a greater surface area per gram of material may positively impact the performance to weight ratio of parts.

Expanded polymeric webs have great utility especially in the consumer products area. An important subsection of expanded polymeric webs is apertured and expanded polymeric webs. Expanded polymeric webs of the apertured type find application in many areas such as topsheets for feminine hygiene and baby care products. The amount of aperturing and the size and shape of the apertures may affect the performance of these films in such applications. The aperturing characteristics are set at the time of production but can change over-time due to alterations in the local polymeric chains caused by external thermal and mechanical forces. As such, the ability to maintain the aperturing characteristics (also called stability) may affect the consumer experience.

One method for producing an expanded and/or apertured polymeric web is via hydroformation. In this process, a flat base polymeric web is impacted with high velocity water while in contact with a typically non-deformable forming structure that may be apertured or non-apertured. The water forces the flat base polymeric web to partially or wholly conform to the positive image of the forming structure. In some areas of the forming structure, the film will also aperture if sufficient force and displacement is allowed. The resulting apertured and expanded polymeric web is then removed from the forming structure.

The amount and openness of the apertured portion of the expanded polymeric web can be quantified by air permeability measurements. Air permeability refers to the volumetric flow rate of air that flows through a given cross-sectional area for a given pressure drop. A higher air permeability generally implies a larger amount of open area and qualitatively tracks the consumer perceived performance of the film product (higher usually being better for fluid acquiring products such as feminine hygiene pads).

In general, the ability to maintain the characteristics of the expanded polymeric web is desired.

### SUMMARY OF THE INVENTION

In one aspect, a product comprises a hydroformed polymeric web consisting of between about 0.1 and about 70 weight percent of a compound comprising nanoparticles, between about 30 and about 99.9 weight percent of a generally melt processable polymer, and between about 0.0 and about 50 weight percent of a compatibilizer. The hydroformed polymeric web has an air permeability that is greater than the air permeability of a hydroformed polymeric web of the melt processable polymer alone. After exposure to compressive forces and elevated temperatures consistent with storage on a roll in an un-conditioned warehouse, also called aging, the polymeric web comprising nanoparticles has improved air permeability relative to the polymeric web without nanoparticles. The % difference in air permeability of the aged polymeric web is equal to or greater than the % difference measured prior to aging.

In another aspect, a product comprises a hydroformed polymeric web consisting of between about 0.1 and about 70 weight percent of a nanoclay, between about 30 and about 99.9 weight percent of a low density polyethylene (LDPE) and linear low density polyethylene (LLDPE) blend, and between about 0.0 and about 50 weight percent of a compatibilizer. The hydroformed polymeric web has an air permeability that is greater than the air permeability of a hydroformed polymeric web of the low density polyethylene and linear low density polyethylene blend alone. After exposure to compressive forces and elevated temperatures consistent with storage on a roll in an un-conditioned warehouse, the polymeric web comprising nanoclay has improved air permeability relative to the polymeric web without nanoclay. The % difference is equal to or greater than the % difference measured prior to aging.

In another aspect, a product comprises a base polymeric web consisting of between about 0.1 and about 70 weight percent of a compound comprising nanoparticles, between about 30 and about 99.9 weight percent of a melt processable polymer, and between about 0.0 and 50 weight percent, of a compatibilizer. The base polymeric web may be hydroformed, vacuum formed or otherwise expanded by means known in the art.

In yet another aspect, a product comprises a ring rolled web material consisting of between about 0.1 and about 70 weight percent of a compound comprising nanoparticles, between about 30 and about 99.9 weight percent of a generally melt processable polymer, and between about 0.0 and about 50 weight percent of a compatibilizer. The ring rolled web material has a machine direction modulus greater than the machine direction modulus of the base web material.

### BRIEF DESCRIPTION OF THE DRAWINGS

While the claims hereof particularly point out and distinctly claim the subject matter of the present invention, it is believed the invention will be better understood in view of the following detailed description of the invention taken in conjunction with the accompanying drawings in which corresponding features of the several views are identically designated and in which:

FIGURE is a schematic plan view of a product according to one embodiment of the invention.

### DETAILED DESCRIPTION OF THE INVENTION

Unless stated otherwise, all weight percentages are based upon the weight of the polymeric web as a whole. All exemplary listings of web constituents are understood to be non-limiting with regard to the scope of the invention.

### I. Definitions

As used herein, the term “expanded polymeric web” and its derivatives refer to a polymeric web formed from a precursor polymeric web or film (equivalently called “base polymeric web” herein), e.g. a planar web, that has been caused to conform to the surface of a three dimensional forming structure so that both sides or surfaces of the precursor polymeric web are permanently altered due to at least partial conformance of the precursor polymeric web to the three-dimensional pattern of the forming structure. In one embodiment the expanded polymeric web is a three dimensional web that comprises macroscopic and/or microscopic structural features or elements. Such expanded polymeric webs may be formed by embossing (i.e., when the forming structure exhibits a pattern comprised primarily of male projections) or debossing (i.e., when the forming structure exhibits a pattern comprised primarily of female depressions or apertures), by tentering, or by a combination of these. Also, such expanded polymeric webs may comprise areas that are fluid pervious (i.e., areas that have been expanded and ruptured forming apertures) and areas that are fluid impervious (i.e., areas that have been expanded without rupture forming surface aberrations). Additional processes for expanding polymeric webs include hydroformation, vacuum formation, and other film expansion methods as are known in the art.

As used herein, the term “hydroformation” and its derivatives refer to the process that uses high-pressure liquid jets to conform the precursor web to the shape of the forming structure and may cause rupture to some parts of the web. More details about hydroformation process can be found in U.S. Pat. No. 4,609,518 issued to Curro et al. on Sep. 2, 1986.

As used herein, the term “vacuum formation” and its derivatives refer to the process that uses vacuum to conform the precursor web to the shape of the forming structure and may cause rupture to some parts of the web.

As used herein, the term “macroscopic” and its derivatives refer to structural features or elements that are readily visible and distinctly discernable to a human having a 20/20 vision when the perpendicular distance between the viewer's eye and the web is about 12 inches.

As used herein, the term “microscopic” and its derivatives refer to structural features or elements that are not readily visible and distinctly discernable to a human having a 20/20 vision when the perpendicular distance between the viewer's eye and the web is about 12 inches.

### II. Expanded Polymeric Webs

In one embodiment, an expanded polymeric web comprises between about 0.1 and about 70 weight percent of a compound comprising nanoparticles. Nanoparticles are discrete particles comprising at least one dimension in the nanometer range. Nanoparticles can be of various shapes, such as spherical, fibrous, polyhedral, platelet, regular, irregular, etc. In another embodiment, the lower limit on the percentage by weight of the compound may be about 1 percent. In still another embodiment, the lower limit may be about 2 percent. In yet another embodiment, the lower limit may be about 3 percent. In still yet another embodiment, the lower limit may be about 4 percent. In another embodiment, the upper limit may be about 50 percent. In yet another embodiment, the upper limit may be about 30 percent. In still another embodiment, the upper limit may be about 25 percent. The amount of the compound present in the polymeric web may be varied depending on the target product cost and expanded polymeric web properties. Non-limiting examples of nanoparticles are natural nanoclays (such as kaolin, talc, bentonite, hectorite, nontmorillonite, vermiculite, and mica), synthetic nanoclays (such as Laponite® from Southern Clay Products, Inc. of Gonzales, Tex.; and SOMASIF from CO-OP Chemical Company of Japan), treated nanoclays (such as organically modified nanoclays), nanofibers, metal nanoparticles (e.g. nano aluminum), metal oxide nanoparticles (e.g. nano alumina), metal salt nanoparticles (e.g. nano calcium carbonate), carbon or inorganic nanostructures (e.g. single wall or multi wall carbon nanotubes, carbon nanorods, carbon nanoribbons, carbon nanorings, carbon or metal or metal oxide nanofibers, etc.), and graphite platelets (e.g. expanded graphite, etc.).

In one embodiment, the compound comprising nanoparticles comprises a nanoclay material that has been exfoliated by the addition of ethylene vinyl alcohol (EVOH) to the material. As a non-limiting example, a nanoclay montmorillonite material may be blended with EVOH (27 mole percent ethylene grade). The combination may then be blended with an LLDPE polymer and the resulting combination may be blown or cast into films. The combination of LLDPE, EVOH and nanoclay materials has been found to possess a substantially higher tensile modulus than the base LLDPE, and substantially similar tensile toughness as LLDPE.

The compound comprising nanoparticles may comprise nanoclay particles. These particles consist of platelets that may have a fundamental thickness of about 1 nm and a length or width of between about 100 nm and about 500 nm. In their natural state these platelets are about 1 to about 2 nm apart. In an intercalated state, the platelets may be between about 2 and about 8 nm apart. In an exfoliated state, the platelets may be in excess of about 8 nm apart. In the exfoliated state the specific surface area of the nanoclay material can be about 800 m²/g or higher. Exemplary nanoclay materials include montmorillonite clay materials and montmorillonite organoclay materials (i.e., montmorillonite clay materials that have been treated with a cationic material that imparts hydrophobicity and causes intercalation), and equivalent clays as are known in the art. Such materials are available from Southern Clay Products, Inc. of Gonzales, Tex. (e.g. Cloisite® series of nanoclays); Elementis Specialties, Inc. of Hightstown, N.J. (e.g.

Bentone® series of nanoclays); Nanocor, Inc. of Arlington Heights, Ill. (e.g. Nanomer® series of nanoclays); and Siid-Chemie, Inc. of Louisville, Ky. (e.g. Nanofil® series of nanoclays).

The expanded polymeric web also comprises between about 30 and about 99.9 percent of a melt processable polymer. The melt processable polymer may consist of any such melt processable thermoplastic material or their blends. Exemplary melt processable polymers include low density polyethylene, such as ExxonMobil LD129.24 low density polyethylene available from the ExxonMobil Company, of Irving, Tex.; linear low density polyethylene, such as Dowlex™ 2045A and Dowlex™ 2035 available from the Dow Chemical Company, of Midland, Mich.; and other thermoplastic polymers as are known in the art (e.g. high density polyethylene—HDPE; polypropylene—PP; very low density polyethylene—VLDPE; ethylene vinyl acetate—EVA; ethylene methyl acrylate—EMA; EVOH, etc). Furthermore, the melt processable thermoplastic material may comprise typical additives (such as antioxidants, antistatics, nucleators, conductive fillers, flame retardants, pigments, plasticizers, impact modifiers, etc.) as known in the art. The weight percentage of the melt processable polymer present in the polymeric web will vary depending upon the amount of the compound comprising nanoparticles and other web constituents present in the polymeric web.

The expanded polymeric web may further comprise a compatibilizer in the range from about 0 to about 50 percent by weight. The compatibilizer may provide an enhanced level of interaction between the nanoparticles and the polymer molecules. Exemplary compatibilizers include maleic anhydride, and maleic-anhydride-modified polyolefin as these are known in the art (e.g. maleic-anhydride-grafted polyolefin).

The nanoclay and compatibilizer may be provided as a masterbatch that may be added to the polymeric web as a single component. Exemplary examples include the NanoBlend™ materials supplied by PolyOne Corp. of Avon Lake, Ohio,

The precursor polymeric web may be formed using any method known in the art, including, without limitations, casting or blowing the polymeric web. Also, the precursor polymeric web may comprise a single layer or multiple layers. The precursor polymeric web may be hydroformed to form an expanded polymeric web. In one embodiment, the precursor polymeric web may be vacuum formed to form an expanded polymeric web.

In one embodiment, the base polymeric web may be processed to become expanded. In this embodiment, the base polymeric web may be pressed between a set of intermeshing plates. The plates may have intermeshing teeth and may be brought together under pressure to deform a portion of the polymeric web.

One plate may include toothed regions and grooved regions. Within the toothed regions of the plate there may be a plurality of teeth. The other plate may include teeth which mesh with teeth of the first plate. When a polymeric web is formed between the two plates the portions of the film which are positioned within grooved regions of the first plate and teeth of the second remain undeformed. The portions of the web positioned between toothed regions of the first plate and the teeth of the second plate are incrementally and plastically formed creating rib-like elements in the polymeric web.

The method of formation can be accomplished in a static mode, where one discrete portion of a web is deformed at a time. Alternatively, the method of formation can be accomplished using a continuous, dynamic press for intermittently contacting the moving web and forming the base material into a formed polymeric web of the present invention. These and other suitable methods for forming the polymeric web of the present invention are more fully described in U.S. Pat. No. 5,518, 801 issued to Chappell, et al. on May 21, 1996. Polymeric webs formed in this manner may be described in U.S. Pat. No. 5,650,214 issued to Anderson et al. on Jul. 22, 1997.

Such an expanded polymeric web may comprise a first region and a second region. When the polymeric web is subjected to an applied elongation along at least one axis the first region may undergo a substantially molecular deformation and the second region may initially undergo a substantially geometric deformation. The expanded polymeric web has greater machine direction and cross machine direction tear propagation resistances than a similarly formed polymeric web of the low density polyethylene alone.

In one embodiment the polymeric web may be expanded by ring rolling the web as is known in the art. As an example, and without limiting the invention, a polymeric web comprising about 56% LLDPE, about 40% CaCO₃, and about 4% treated nanoclay particles was ring rolled in a ring rolling apparatus with a roll pitch of 0.060 inches (about 0.15 mm) and a depth of engagement of about 0.075 inches (about 0.19 mm). The machine direction modulus of the expanded web was found to be about 50% greater than the modulus of a similarly ring rolled web without the nanoclay particles.

The air permeability of the expanded polymeric web with nanoparticles may be greater than the air permeability of an expanded polymeric web consisting of the melt processable polymer alone. The air permeability of the polymeric webs is tested by placing a sample of a web (noting direction of orientation of 3-D structures forming the apertures) over an aperture and drawing air through the web and the aperture by creating a known level of negative pressure on the non-material side of the aperture. The air flow through the polymeric web at a known pressure drop in cubic feet per minute (CFM) is representative of the air permeability of the web. A comparison of relative air permeabilities of distinct webs may be conducted by testing sample of the web using the same aperture and the same pressure differential and then comparing the CFM values for each of the webs. The web may be tested using a Tex Test model FX 3300 permeability tester, available from Tex Test, Ltd., of Zurich, Switzerland.

Surprisingly, applicants have found the air permeability of an expanded polymeric web may be improved by 10% at a given pressure drop with the incorporation of nanoparticles to the polymeric web. Additionally, the addition of nanoparticles yields an air permeable structure which is more stable over time with regard to air permeability. After exposure to compressive forces and elevated temperatures consistent with storage on a roll in an un-conditioned warehouse, also called aging, the expanded polymeric web comprising nanoparticles has improved air permeability relative to the expanded polymeric web without nanoparticles. The % difference is equal to or greater than the % difference measured prior to aging.

The air permeability of an expanded polymeric web may decrease over time as the web ages. The addition of nanoparticles to the web may provide a means of slowing the loss of air permeability in a polymeric web. Test results have indicated an improvement in the air permeability of aged expanded polymeric webs comprising nanoparticles relative to an expanded polymeric web without nanoparticles of between 17% and about 37% over time, with the improvement increasing over time.

In one embodiment, the expanded polymeric web with nanoparticles has a compression aged air permeability that is greater than the compression aged permeability of an expanded polymeric web without nanoparticles. Compression aged air permeability may be determined by preparing 18 samples of the polymeric web each sample about 4 inches (10 cm) square. The samples are stacked and subjected to a compressive force for about 0.5 psi for a period of about 17 hours. The ten samples from the center of the stack are then removed and the air permeability of each of these samples is then tested as set forth above.

In one embodiment, the expanded polymeric web comprises an elevated temperature aged air permeability that is greater than the elevated temperature aged air permeability of an expanded polymeric web of the melt processable polymer alone. The elevated temperature aged air permeability may be determined by preparing 18 samples of the film material each sample about 4 inches (10 cm) square. The samples are stacked and subjected to a compressive force for about 0.5 psi for a period of about 17 hours at a temperature of about 60 C. The ten samples from the center of the stack are then removed and the air permeability of each of these samples is tested as set forth above.

Other materials may be added to the precursor polymeric web. In one embodiment, the precursor polymeric web may comprise calcium carbonate (CaCO₃) in an amount of between about 5% and about 70% of CaCO₃.

The machine direction tensile modulus is the tensile modulus of the material measured along the direction of film travel during the manufacturing of the film. The cross machine direction tensile modulus of the polymeric web is the tensile modulus measured across the direction of web travel during the manufacturing of the polymeric web. Each of the machine direction and cross machine direction tensile modulus may be determined by the ASTM D 882-02 Standard Test Method for Tensile Properties of Thin Plastic Sheeting using 1 inch wide by 6 inch long rectangular strips, 2 inch clamp separation, 20 inch per minute test speed and no extensometer.

In one embodiment, the precursor polymeric web comprising at least the nanoparticles comprised a greater tear resistance than the tear resistance of the precursor polymeric web without the nanoparticles. The addition of CaCO₃ to a precursor polymeric web comprising a melt processable polymer and nanoparticles may further improve the propagation tear resistance and tensile modulus in each of the machine and cross directions. In this embodiment, propagation tear resistance of the respective polymeric webs may be determined using the ASTM D1922-05 Standard Test Method for Propagation Tear Resistance of Plastic Film and Thin Sheeting by Pendulum Method.

### Example 1

A 1 mil (0.0254 mm) cast film of linear low density polyethylene and low density polyethylene in a ratio of about 70:30 is prepared together with a 1 mil (0.0254 mm) thick cast film of the same ratio of polymers together with 10% by weight of NanoBlend™ 2101 which comprises between 38 and 42% organoclay particles. Each of the cast films is hydroformed yielding an apertured and expanded film. The air permeability of each expanded polymeric web is tested immediately after formation and the nanocomposite film is found to have an air permeability about 10% (i.e., about 50 CFM) higher than that of the expanded polymeric web comprising no nanoclay particles. After one week of aging at ambient temperature and without a compressive load, the expanded polymeric web comprising nanoclay particles has an air permeability about 17% greater than that of the expanded polymeric web comprising no nanoclay particles. After stacked compressive aging at ambient temperature, the expanded polymeric web comprising nanoclay particles has an air permeability about 24% greater than that of the expanded polymeric web comprising no nanoclay particles. After stacked compressive aging at an elevated temperature of about 60 C, the expanded polymeric web comprising nanoclay particles has an air permeability about 37% higher than that of the expanded polymeric web comprising no nanoclay particles.

### Product Examples

The expanded polymeric web materials of the invention may be utilized in any application where an apertured web, an expanded web or an elastic-like web would be beneficial. The requirements of the intended use may be associated with the particular composition of the web and also with the method of expanding the web material.

Exemplary uses include, without limiting the invention, an apertured fluid transfer topsheet as part of a diaper, training pant, feminine hygiene product, adult incontinence product or any product where fluid transfer through a web material is a consideration. Web materials having first and second regions with different response to applied stress may be utilized in applications where some degree of elasticity, web drape, or both are desired. Exemplary uses include, without being limiting, diaper leg cuffs and side panels, training pant panels, feminine hygiene product edge portions, and adult incontinence panels.

In one embodiment illustrated in FIGURE, an absorbent article 10 comprises a chassis 12. The chassis 12 comprises a fluid permeable topsheet 14 formed from the expanded polymeric web material comprising nanoparticles described above. The article may optionally comprise a fastening system, barrier cuffs, gusseting cuffs, and may be configured such that the chassis comprises front and/or back ears. Elements of the article may comprise a lotion as is known in the art. Exemplary absorbent articles include, without being limiting, diapers, feminine hygiene garments, adult incontinences articles, training pants, and diaper holders. Without limiting the invention, absorbent article structures that may comprise an expanded polymeric web topsheet as described herein are described in U.S. Pat. Nos. 3,860,003; 5,151,092; 5,221,274; 5,554,145; 5,569,234; 5,580,411; 4,589,876 and 6,004,306.

In another embodiment, an absorbent article may comprise an expanded polymeric film having first regions and second regions as set forth above. Such films may be used as a portion of absorbent articles including without being limiting, diapers, feminine hygiene garments, adult incontinences articles, training pants, and diaper holders. Such films may be used to impart an elastic-like nature to at least a portion of an article.

In another embodiment, a disposable absorbent product may comprise a ring rolled web comprising nanoclay particles and optionally comprising CaCO₃. The ring rolled web material may be utilized as an element of the product to provide an extensible element without the need to include rubber compounds in the element. The material may be ring rolled using the apparatus and methods for ring rolling films as these are known in the art.

The expanded polymeric web materials described may be utilized as elements of other products as well as the uses set forth above. Exemplary uses for the expanded polymeric webs include, without limiting the invention, film wraps, bags, polymeric sheeting, outer product coverings, packaging materials, and combinations thereof.

The expanded polymeric web materials may be incorporated into products as direct replacements for otherwise similar web materials which do not comprise nanoparticles.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would have been obvious to those skilled in the art that various other changes and modifications can be made without departing from the spirit and scope of the invention. It is therefore intended to cover in the appended claims all such changes and modifications that are within the scope of the invention.

1. A product comprising an expanded polymeric web, the expanded polymeric web comprising:
a) between about 0.1 and about 70 weight percent, of a compound comprising nanoparticles,
b) between about 30 and about 99.9 weight percent of a generally melt processable polymer, and
c) between about 0.0 and about 50 weight percent of a compatibilizer.

2. The product of claim 1 wherein the product comprises a disposable absorbent product.

3. The product of claim 2 wherein the expanded polymeric web material comprises a fluid pervious topsheet.

4. The product of claim 3 wherein the product comprises a diaper.

5. The product of claim 3 wherein the product comprises a feminine hygiene product.

6. A product according to claim 1 wherein the web material has been expanded by hydroformation.

7. The product according to claim 1 wherein the web material comprises a hydroformed base polymeric web, and has an air permeability that is greater than the air permeability of an expanded polymeric web of the melt processable polymer alone.

8. The product according to claim 1 wherein the web material has been expanded by vacuum formation.
